(19) United States
(12) Patent Application Publication
(10) Pub. No.: US 2005/0074868 A1
(43) Pub. Date: Apr. 7, 2005

(54) Method of genomic analysis

(76) Inventors: Nicholas Schork, San Diego, CA (US); Jeremy Shaun Lanehbury, Salt Lake City, UT (US)

Correspondence Address: CALFEE HALTER & GRISWOLD, LLP, 800 SUPERIOR AVENUE, SUITE 1400, CLEVELAND, OH, 44114, US

(21) Appl. No.: 10/483,143

(22) PCT Filed: Jul. 8, 2002
(86) PCT No.: PCT/US02/21377

(30) Foreign Application Priority Data
Jul. 6, 2001 (GB) 0116565.3

(51) Int. Cl. C12M 1/00

(52) U.S. Cl. 435/285.1

(57) ABSTRACT

The present invention relates to a method for identifying genomic regions comprising one or more genes that affect a biological phenotype such as the level of lymphocyte subpopulations. The present invention also relates to isolated genomic regions identified using the method of the present invention, one or more genes contained in the genomic regions and methods for detecting the presence of the one or more genes in an individual.

The present invention relates to a method for identifying genomic regions comprising one or more genes that affect a biological phenotype such as the level of lymphocyte subpopulations. The present invention also relates to isolated genomic regions identified using the method of the present invention, one or more genes contained in the genomic regions and methods for detecting the presence of the one or more genes in an individual.

A properly functioning immune system is a necessary component of human survival. Cell populations which comprise the immune system and which are responsible for functional responses carry surface structures which can be recognised by monoclonal antibodies (tabs). The major lymphocyte populations identified in this way are CD4 and CD8+ T cells, B cells and natural killer (NK) cells. These subsets of lymphocytes play a vital role in defence against tumours, bacteria, viruses and other parasites, and in the pathology of autoimmune diseases. Levels of these cells and derived measures such as CD4:CD8 ratio vary between humans and a significant proportion of this variation appears to be due to genetic differences. The best example of the clinical relevance of lymphocyte subpopulation variation is in the prognosis and monitoring of the acquired immunodeficiency syndrome (AIDS). Patients with initially high levels of CD4+ T cells and high CD4:CD8+ T cell ratios show slower progression to AIDS than patients with lower values. Other viral infections such as cytomegalovirus, Epstein-Barr and influenza are associated with perturbations of the CD4:CD8+ T CELL ratio. Chronic immune-mediated or inflammatory conditions such as allograft rejection, graft-versus-host disease, Sjogren's syndrome and polymyalgia rheumatica also show abnormalities of T cell levels and ratios while rheumatoid arthritis (RA) and systemic lupus erythematosus (SLE) show perturbations of B cell populations. The extent to which genetic variation in lymphocyte subset levels accounts for a proportion of the risk for developing the aforementioned diseases is unknown.

B and T cells form the major components of the adaptive arm of the immune system. In recent years there has been an explosion in the understanding of the molecular basis of many of the components of the immune system. Studies of animal models and individuals who have naturally occurring deficiencies of the immune system along with improvements in molecular biology techniques have led to the identification of many of the genes which are involved in the development and function of the immune system. These include genes responsible for lymphocyte subset differentiation, cell function, immunity to various micro-organisms, and lymphocyte activation regulation. Such studies reveal a complex set of developmental pathways which result in the generation of a large repertoire of B and T lymphocytes, natural killer cells, monocytes, macrophages and dendritic cells. Together, these provide the individual with the capability of mounting successful adaptive and innate immune responses. There are a number of genes characterised whose lack of function result in a total absence of a particular cell type and thus result in a number of immune deficiencies in humans. In contrast, there is little knowledge of how genes may play a role in modulating the actual levels of the individual cell populations whilst still maintaining a functional balance of the immune system.

The extent to which polymorphic genes account for the variation in levels of functional populations of lymphocyte which comprise the human immune system has not been investigated substantially. Several studies have concentrated on T cell populations showing that variation in the important clinical parameters of CD4 and CD8+ T cell levels and CD4:CD8 ratio are significantly heritable. It has also been suggested by segregation analysis that CD4:CD8 ratio and CD4 and CD8+ T cell levels are under major recessive gene control.

Studies of the impact of genetic variation on the functioning of the immune system have up to now largely concentrated on qualitative issues of response and non-response. Most studies have been conducted in the mouse, and experimental manipulation of the murine genome using “knock out” and transgenic technologies has enabled insight to be gained in systems which are largely driven to phenotypic extremes. Recently, improvements in the mapping of QTL's (quantitative trait linkage analysis) for complex traits have been described which enable rapid interval mapping between inbred mouse strains (Grupe 2001).

There is a need for an efficient method for identifying genomic regions that affect the biological phenotype of an individual. There are numerous biological phenotypes that have a genetic component including weight, height, skin colour, hair colour etc. A particularly preferred biological phenotype is the level of subpopulations of lymphocytes. The level of the various cell populations that comprise the immune system vary and this variation can lead to differences in an individual's immune response. By identifying the genomic regions, which contain one or more genes that affect the level of subpopulations of lymphocytes, the ability of an individual to raise a particular immune response can be determined by determining the presence of the one or more genes.

Previous methods for identifying genomic regions that have an affect on a biological phenotype have mainly been based on performing linkage analysis on paired siblings. Such methods have a number of disadvantages including the necessity ofor having to use large sample sizes.

The present invention overcomes at least some of the disadvantages of the previous methods and provides an efficient method for identifying genomic regions comprising one or more genes that affect a biological phenotype.

The present invention provides a method for identifying a genomic region comprising one or more genes affecting a biological phenotype, comprising performing linkage analysis on one or more extended families wherein the total number of individuals is at least 50.

By performing the linkage analysis in extended families statistically significant data can be obtained using less individuals than in the prior art methods using paired siblings.

The term “genomic region” as used herein refers to a region from the genome of an individual. As the genome of eukaryotic organisms is made up of chromosomes the term “chromosomal region” is also used herein to refer to a genomic region. The genomic region is preferably less than 6 cM in size, more preferably about 5 cM or less. As will be appreciated by those skilled in the art the size of the genomic region identified using the method of the present invention will vary depending on the number of markers used in the linkage analysis and the spacing of the markers on the genome and the number of extended families.

The term “a biological phenotype” refers to any measurable phenotype which has a genetic component including weight, height, skin colour, hair colour, level of chemokines, blood pressure, arterial blood velocity, auditory accuity, visual accuity, cognitive variables, IQ, bone density, fasting glucose levels, tissue insulin resistance, etc. Preferred phenotypes include the level of lymphocytes and other white blood cells such as monocytes, macrophages, dendritic cells and granulocytes, as well as the level of red blood cells or platelets. A particularly preferred biological phenotype is the level of subpopulations of lymphocytes, especially the level of CD4+ T cells, the level of CD8+ T cells, the level of B cells, the level of NK cells or the ratio of CD4+ to CDS+ T cells. It is most preferred that the biological phenotype is the ratio of CD4+ to CD8+ T cells or the level of CD4+ T cells.

The biological phenotype can be measured by using standard techniques. In particular, when the biological phenotype is the level of lymphocyte subpopulations, then the subpopulations can be measured using labelled antibody molecules having specificity for a particular subpopulation. Such methods are described in the examples below.

The one or more genes affecting the biological phenotype cause a measurable change in the biological phenotype. The change in the biological phenotype may be enhanced by one or more other genes which may or may not be comprised within the same genomic region. The biological phenotype may be affected in any measurable way. For example where the biological phenotype is the level of CD4+ T cells, the one or more genes may increase or decrease the level of CD4+ T cells in an individual, or lead to a reduction or increase in the level of fluctuation of CD4+ T cell levels in an individual over a particular time period or, for example, following a challenge with an immunogen.

The linkage analysis performed can be performed using any standard method. For example, the linkage analysis can be qualitative analysis but is preferably quantitative trait linkage (QTL) analysis. There are numerous methods for performing such analysis well known to those skilled in the art including the Genehunter, Solar etc. packages. Preferably QTL analysis is performed using the SIBPAL2 program implemented in the SAGE package (SAGE, 2001, Statistical Analysis for Genetic Epidemiology, Beta 4.0-7, available from the Department of Epidemiology and Biostatistics, Rammelkamp Center for Education and Research, MetroHealth Campus, Case Western Reserve University, Cleveland) or any subsequently updated version.

The term an “extended family” as used herein means a family comprising at least 2 generations and at least 5 siblings in the youngest generation. Preferably the extended family comprises at least 3 generations.

It has been found that by performing linkage analysis on one or more extended families wherein the total number of individuals is at least 50 that it is possible to identify genomic regions containing one or more genes that affect a biological phenotype.

It is preferred that the total number of individuals in the one or more extended families is at least 100, more preferably at least 150 and most preferably at least 300. As one skilled in the art will understand the greater the number of individuals analysed the greater the confidence in the data obtained. It is therefore desirable to perform the linkage analysis on as many individuals in extended families as possible; however, the minimum number of individuals in extended families required to enable one skilled in the art to identify genomic regions comprising one or more genes affecting a biological phenotype is at least about 50.

Preferably the linkage analysis is performed on at least 4 extended families. It is preferred that the linkage analysis is performed on at least 10 extended families, more preferably at least 17 extended families and most preferably at least 30 families. As one skilled in the art will understand the greater the number of extended families analysed the greater the confidence in the data obtained.

The method of the present invention can be performed on genomic material (i.e. at least part of the genome) of any organism including eukaryotic organisms and prokaryotic organisms. Preferably the method of the present invention is performed on genomic material of a mammal, most preferably a human.

In a preferred embodiment of the present invention the method of the present invention additionally comprises performing fine mapping techniques on the genomic region identified using the method of the present invention. Numerous fine mapping techniques are well know to those skilled in the art and include quantitative transmission disequilibrium test (QTDT) analysis and the case-control approach.

Preferably the fine mapping technique used is QTDT analysis. QTDT analysis is preferably performed using the ASSOC program of the SAGE package (supra) although any suitable program for performing QTDT analysis can be used.

The present invention also provides an isolated genomic region identified using the method of the present invention.

The isolated genomic region is a region of the genome which has been isolated from an individual. The genomic region can be isolated from the genome using standard molecular biological techniques well known to those skilled in the art.

Preferred isolated genomic regions according to the present invention are listed in Table 5. The positions of the genomic regions are given as cM positions on the chromosome. These positions are obtained from the Marshfield Clinical genetic map, which is integrated with the physical genome map (Broman et al., Am. J. Hum. Genet., 63, 861-869, 1998, and Yu et al., Nature, 409, 951-953, 1998). Accordingly, one skilled in the art can identify the ends of the genomic regions.

In a preferred embodiment the isolated genomic region is located at 197 to 218 cM on chromosome 1. This genomic region was found using the method of the present invention and affects the level of CD8+ T cells in a human.

In a further preferred embodiment the isolated genomic region is flanked by markers D4S405 and D4S2363 on chromosome 4. The sequence of markers D4S405 and D4S2363 is given in the Marshfield Clinical genetic map, which is integrated with the draft genome sequence according to Golden Path April 2001 freeze (http://genome.ucsc.edu). This genomic region was found using the method of the present invention and affects the ratio of CD4 to CD8+ T cells in a human.

In a preferred embodiment the isolated genomic region is located at 90 to 110 cM on chromosome 18. This genomic region was found using the method of the present invention and affects the level of CD4+ T cells in a human.

The present invention also provides a gene contained in the genomic region of the present invention, wherein the gene affects the biological phenotype. The gene can be identified by performing fine mapping techniques as discussed above. In a particular embodiment of the present invention it has been found that the isolated genomic region located at 90 to 110 cM on chromosome 18 contains 5 genes, namely TCF-4 (56881247-57294117, nucleotide positions in draft sequence according to Golden Path April 2001 freeze (http://genome.ucsc.edu/), IDDM6 (Merriman et al., Diabetes, 50 184-194, 2001), Bc1-2α (65421315-65641279, nucleotide positions as indicated above), Bc1-2β (65613484-65614394, nucleotide positions as indicated above) and RANK, all of which have been implicated in T cell function and/or autoimmune disease risk. In particular, Bc1-2α is recognised as having a profound effect on lymphocyte survival and through associated variation, could be a strong candidate for determining heritable differences in lymphocyte subset levels. Bc1-2α is therefore considered to be a gene which affects the biological phenotype of CD8+ T cell levels.

The present invention also provides the encoded product of the gene of the present invention. The use of the encoded product of the gene of the present invention in therapy is also provided by the present invention.

Preferably the gene of the present invention is located within ±1 cM of a marker of the biological phenotype, wherein the marker has a p-value of less than 0.05 as calculated by QTDT analysis. Preferably the marker is any one of the markers listed in Table 6 below. It is further preferred that the p-value is less than 0.01. It is also preferred that the p-values are determined after the Bonferroni correction.

The present invention also provides the encoded product of the gene of the present invention. The use of the encoded gene of the present invention in therapy is also provided by the present invention.

The present invention also provides an assay for detecting an individual's risk of developing a disease, for diagnosis of a disease, for prognosis of a disease or for determining the efficacy or toxicity of a treatment comprising determining the presence of the gene of the present invention in the individual's genome.

As will be clear to one skilled in the art, the specific use of the present invention will depend on the phenotypic effect associated with the gene. For example, if the gene is associated with a higher ratio of CD4+ to CD8+ T cells, then the presence of the gene causing the higher ratio, will indicate a decreased risk of an individual infectde with HIV developing AIDS quickly, (i.e. being a fast progressor (see Amadori et al., Immunology today, 17, 414-417, 1996). Genes which cause the phenotypic effect of increased CD4+ T cell levels or reduced CD8+ T cell levels can also be used to indicate a decreased risk of an individual infected with HIV developing AIDS quickly.

In some other situations the presence of the genomic region or the gene in an individual, alone or in combination with other data, will be indicative of the individual having a particular disease.

The presence of the genomic region or the gene in an individual, alone or in combination with other data, may also enable or assist a practitioner to determine the prognosis of a particular disease or to determine the efficacy or toxicity of a particular treatment. In some situations, it may be possible to determine the optimal treatment of a particular disease once it has been determined whether the genonic region is present or absent. The use of specific known genetic risk factors for determining an individual's risk to a particular disease is well known to those skilled in the art. Furthermore, the use of specific known genetic sequences to determine the efficacy or toxicity of a drug are also known in the art. Alternatively. The encoded product of the gene of the present invention can be detected in the assay. Suitable labeled antibody molecules having affinity for the encoded product can be used in such an assay.

Preferably the assay of the present invention is performed by contacting a sample of the individual's genomic material with a labelled probe specific for the gene of the present invention. Preferably the probe is a labelled nucleic acid probe. The use of labelled nucleic acid probes as well as the manufacture of the probes is well known to those skilled in the art.

The present invention also provides a method for identifying an agonist or antagonist of the product encoded by the gene of the present invention. Suitable methods for identifying such agonists and antagonists include screening methods wherein libraries of compounds are screened. Candidate agonists and antagonists may be isolated from, for example, cells, cell-free preparations, chemical libraries, or natural product mixtures. These candidate compounds may be natural or modified substrates, ligands, enzymes, receptors or structural or functional mimetics. For a suitable review of such screening techniques, see Coligan et al., Current Protocols in Immunology 1(2):Chapter 5 (1991).

Antagonists and agonists identified by the method described above can be used to reduce or enhance the effects of the gene of the present invention, respectively. Accordingly, the agonists and antagonists can be used in a method of treatment or prophylaxis of a disease caused, at least in part, or prevented, at least in part, by the product encoded by the gene of the present invention.

The present invention relates to the use of a nucleic acid molecule encoding the product of the gene of the present invention in a method of gene therapy for treating or preventing a disease. The present invention also relates to the use of the product encoded by the gene of the present invention in the manufacture of a medicament for the treatment or prevention of a disease. Preferably the disease is associated with the phenotypic effect caused by the gene. Suitable diseases include AIDS, cancer, autoimmune diseases and inflammatory diseases.

The present invention also provides the use of the gene of the present invention in an assay for identifying a biochemical pathway that is involved in affecting a biological phenotype.

By identifying the biochemical pathway involved in the biological phenotype it will be possible to identify other parts of the pathway that can be blocked or enhanced in order to affect the phenotype. For example, where the phenotype is a disease, by blocking a part of the biochemical pathway giving rise to the disease, it is possible to prevent the disease. The identification of the biochemical pathway is particularly important as some parts of the pathway may be easier to block/enhance than other parts.

In a particularly preferred embodiment of the present invention there is provided a method of predicting the speed of development of AIDS in an individual infected with HIV comprising detecting the presence of one or more genes, which affect the ratio of CD4+ T cells to CDS+ T cells, in the genomic region of chromosome 4 that is flanked by markers D4S405 and D4S2363.

Preferably the method comprises:
taking a cell sample from the individual; and determining the presence of the one or more genes, which increase the ratio of CD4+ T cells to CD8+ T cells, wherein the presence of the one or more genes is indicative of a genetic predisposition to a high CD4:CD8 ratio which reduces the speed of development of AIDS in the individual.

Individuals that develop AIDS slowly are termed long-term non-progressors and individuals that develop AIDS quickly are termed fast-progressors (see Amadori et al., Immunology Today, 17, 414-417, 1996).

As will be appreciated by those skilled in the art the particularly preferred method of the present invention can be performed wherein the presence of one or more genes, which provide a high CD4+ T cell level or a low CD8+ T cell level, can be used to predict the speed of development of AIDS in an individual infected with HIV.

The present invention will now be described in detail with reference to the following example and Figure. It will be appreciated that the invention is described by way of example only and modification of detail may be made without departing from the scope of the invention as defined in the appended claims.

FIG. 1 shows the total genetic variance for CD4+ /CD8+ levels on chromosome 4.

### EXAMPLE

Methods and Materials

Subjects

Subjects were recruited through the Utah Genetics Reference Project (UGRP) based in Salt Lake City, Utah, United States. Originally these consisted of healthy Caucasoid three generation extended families with at least seven siblings in the third generation but grandparents were not available in every case. Thirty-five of these families have been used to generate linkage maps as part of the CEPH Human Genome Mapping Project (Dib et al., Nature, 380, 152-154, 1996). Each family visited clinic at the University of Utah Medical Centre where a thorough clinical examination was carried out and a questionnaire designed to assess the health status of the family members administered. Exclusion criteria include immune-mediated disease (rheumatoid arthritis, type I diabetes, organ specific autoimmune disease and allergic asthma), current infection, past or active leulcaemia or lymphoma and past or present radio- or chemotherapy. The families used in this study were CEPH pedigrees 1420, 1344, 1350, 1377, 1362, 1418, 1408, 1345, 1340, 1477, 1349, 1421, 1346, 1334, 1424, 1375 and 1358.

Flow Cytometry

Fasting venous blood samples (5 ml) were drawn into glass vacutainers containing sterile preservative-free heparin (Becton Dickinson, Franklin Lakes, N.J.) and analysed within 2 hours. Samples were taken at 7 am to minimise any circadian fluctuation in leukocyte population levels.

Fluorescent-conjugated antibody mixtures were added to 100 □l of whole blood, incubated at 22° C. for 15 minutes and then processed using the ImmunoPrep reagent system (Beckman Coulter, High Wycombe UK), which includes a 1% paraformaldehyde fixation step. The cells were then suitable for flow cytometry for up to 5 days when kept at 4 □C. All monoclonal antibodies used were directly conjugated mouse IgG1 anti human and were: IgG1-FITC 679.1Mc7, IgG1-PE 679.1Mc7, IgG1-Cy5 679.1Mc7, CD3-Cy5 UCHT1, CD45-Cy5 J.33 (All Immunotech/Beckman Coulter, High Wycombe UK) and CD4-FITC SK3, CD8-PE SK1, CD16FITC NKP15, CD19PE 4G7, CD56PE MY31 (Becton Dickinson, Oxford, UK).

Stained cells were flown overnight at 4 □C at cabin pressure to London for analysis. Flow cytometry was carried out no more than 40 hours after cell fixation and was performed using a Coulter EPICS-XL I set up for 3 colour detection and XL system II software (Beckman Coulter, High Wycombe UK). The cytometer was calibrated daily using ImmunoCheck beads (Beckman Coulter, High Wycombe UK) in order to maintain a half peak coefficient of variation of less than 2 for each channel. Lymphocytes were gated according to their forward and side scatter properties and 5000 events recorded. The lymphocyte purity of this region was calculated using a CD45 specific monoclonal antibody and was >99% in each case. Positive thresholds were defined by quadrant regions, which enclosed the negative control population on each axis. FlowCount (Beckman Coulter, High Wycombe UK) beads were used to estimate the concentration of lymphocytes in quadruplicates of each sample and the absolute number for each lymphocyte subset was calculated from this.

Simple Tandem Repeat Marker Genotyping

Genotype data on the 17 extended pedigrees used in this study were available from the CEPH database (http://www.cephb.fr/cephdb/). Genetic markers were exclusively autosomal short tandem repeats and positions were assigned using the Marshfield Genetic map (supra). The average intermarker distance was 5 cM. A detailed list of the markers used were obtained from the CEPH database

Heritability Analysis

Table 1 summarises the number of spouse-spouse, parent-offspring and sibling-sibling pairs available for analysis of immune phenotypes. Estimates of familial correlations and their standard deviations were obtained after traits were adjusted by covariates such as age and sex using REGC. Heritability was calculated from the familial correlations using ASSOC assuming parent-offspring and sibling-sibling correlations are the same.

Quantitative Trait Linkage Analysis

QTL analysis was carried out using the SIBPAL2 programme implemented in the SAGE package. The W2 variation of the revised Haseman-Elston regression approach was followed. This method uses a weighted combination of squared trait difference and squared mean-corrected trait sum. Weights are chosen proportional to the inverse residual variance of the squared differences and sums. In our dataset the W2, product and difference variants of the revised Haseman-Elston method, gave comparable results while the W3 method always returned more significant p values (data not shown).

Simulation of Empirical D Values

Quantitative Transmission Disequilibrium Test (OTDT) Analysis

The method proposed by George et al. [1999] was used to conduct QTDT analysis. This test detects linkage in the presence of association. The maximum likelihood This test detects linkage in the presence of association. The maximum likelihood estimates of the parameters and the standard errors of the estimates are computed by numerical methods. These procedures are implemented in the program ASSOC of the S.A.G.E. [1998] software package.

Results

Distributions and Covariates

Table 2 shows distributions of values including numbers studied, means, standard deviations, maximum and minimum values for the 6 lymphocyte subpopulation parameters studied in the 17 CEPH pedigrees. All values were within the expected ranges based on previously published data. Mean age of subjects was 42.5 years (SD 18.7, youngest 18 years, eldest 90 years). Variation in several of the traits under study is highly correlated and Pearson correlation coefficients are summarised in Table 3. Relationships may be divided into trivial and material. For example the lymphocyte count variable is largely additively composed of CD4+ T cell, CD8+ T cell, CD19 B cell and NK cell numbers. This leads to highly significant correlations (each p=0.0001). Other correlations, however, such as CD19 B cells with both CD4 and CD8+ T cells (0.57 and 0.44, both p=0.0001) and NK cells with CD4 and CD8+ T cells (0.26 and 0.57, p=0.01 and 0.0001, respectively) are likely to be functionally based. Age was negatively correlated with CD19 B cell level (p=0.002) and nonsignificantly related to lymphocyte count but had no significant effect on the other phenotypes in these families.

TABLE 1
Summary of Utah Genetic Reference
Project (CEPH) families used for linkage
analysis. Number of pairs corresponding to available trait data.

| | | | Parent- |
|---|---|---|---|
| Traits | Spouse | Sibling | Offspring |
| Lymphocyte Count, CD4, CD8, CD4:CD8 | 18 | 385 | 225 |
| CD19 | 17 | 374 | 211 |
| NK | 7 | 209 | 119 |

TABLE 2
Summary statistics on variables measured in 17 Utah Genetic
Reference Project (CEPH) families used for linkage analysis.
With the exception of CD4:CD8 ratio and age, values
are expressed in millions per ml.

| | | | Standard | Minimum | Maximum |
|---|---|---|---|---|---|
| Variable | Number | Mean | deviation | value | value |
| Lymphocyte count | 142 | 1.29 | 0.45□ | 0.18 | 2.63 |
| CD19 B cells | 138 | 0.16 | 0.08 | 0.02 | 0.41 |
| NK cells | 83 | 0.06 | 0.04 | 0.001 | 0.21 |
| CD4+ T cells | 142 | 0.59 | 0.24 | 0.08 | 1.4 |
| CD8+ T cells | 142 | 0.36 | 0.16 | 0.05 | 0.94 |
| CD4:CD8 ratio | 142 | 1.81 | 0.83 | 0.19 | 6.25 |
| Age | 100 | 42.54 | 18.76 | 18 | 90 |

Heritabilities

Estimates of familial correlations and their standard deviations when traits are adjusted by the covariates age and sex are given in Table 3. Heritability estimates are shown in Table 4. All 6 of the immune traits studied are under significant genetic control with CD8+ T cell level the most heritable trait.

TABLE 3
Pearson correlation coefficients for variables measured in 17
Utah Genetic Reference Project (CEPH) families used for
linkage analysis. Upper value is correlation coefficient,
lower is correlation p value.

| | Lymphocyte | CD19 B | NK | CD4+ T | CD8+ | CD4:CD8 | |
|---|---|---|---|---|---|---|---|
| Variable | count | cells | cells | cells | T cells | ratio | Age |
| Lymphocyte | | 0.69 | 0.45 | 0.87 | 0.80 | 0.02 | −0.18 |
| count | | 0.0001 | 0.0001 | 0.0001 | 0.0001 | 0.80 | 0.07 |
| CD19 B cells | | | 0.18 | 0.57 | 0.44 | 0.07 | −0.31 |
| | | | 0.10 | 0.0001 | 0.0001 | 0.40 | 0.002 |
| NK cells | | | | 0.26 | 0.57 | −0.24 | 0.04 |
| | | | | 0.01 | 0.0001 | 0.02 | 0.68 |
| CD4+ T cells | | | | | 0.47 | 0.40 | −0.15 |
| | | | | | 0.0001 | 0.0001 | 0.12 |
| CD8+ T cells | | | | | | −0.47 | −0.10 |
| | | | | | | 0.0001 | 0.30 |
| CD4:CD8 | | | | | | | 0.08 |
| ratio | | | | | | | 0.43 |

TABLE 4
Heritability estimates derived from
spouse-spouse, parent-offspring and
sibling-sibling correlations with
corresponding variances below each estimate in
brackets. Trait data are adjusted for the covariates age and sex.

| Traits | Heritability |
|---|---|
| Lymphocyte count | 0.6084 |
| | (0.0437) |
| CD19 | 0.1778 |
| | (0.0561) |
| NK | 0.3592 |
| | (0.0676) |
| CD4 | 0.5291 |
| | (0.0548) |
| CD8 | 0.7787 |
| | (0.0416) |
| CD4:CD8 | 0.4946 |
| | (0.0612) |

Quantitative Linkage Analysis

Table 5 gives a summary of the most significant results of a whole genome scan for the 6 quantitative immune phenotypes studied. Nominal p values (p<0.01) are shown for linkages unadjusted for covariates age and sex. Corresponding nominal p values when linkages were adjusted for both age and sex are also given. Empirical p values calculated by simulation are given for the most significant model in each case. The cM points given in Table 5 are Marshfield Clinical Genetic map points. The Marshfield Clinical Genetic map is integrated with the physical genome map.

Twenty-nine unadjusted phenotype-region combinations showed nominal evidence of genetic linkage at this significance level. Two of these, CD8+ T cell level chromosome 18 region 114 cM and lymphocyte count chromosome 2 region 248 cM were no longer significant when adjusted for covariates while p values for most other linkages became more significant or were unchanged.

A major QTL accounting for 30% of the genetic variance of CD4:CD8 ratio maps to the centromeric region of chromosome 4 between 53 and 86 cM (flanked by D4S405 and D4S2363) maximising at 59 cM (nominal covariate adjusted p=0.00000019; empirical covariate adjusted p<0.000001). A QTL for CD4 levels maps to chromosome 18 q between 90 and 110 cM maximising at 102 cM (nominal covariate unadjusted p=0.0000058). This QTL explains 3% of the CD4 trait genetic variance. QTLs for the highly correlated measures of lymphocyte count and CD19 B cell count co-localised with the CD4 level QTL at 18q21 (nominal covariate unadjusted p=0.00032 and nominal covariate adjusted 0.0021, respectively). This suggests the presence of a QTL involved in determining CD4+ T cell and B cell levels (and reflected indirectly by the lymphocyte count) but which does not play a major role in CD8+ T cell levels. Various QTLs for CD8, CD4, NK, CD19 and lymphocyte levels were significant at the p=0.001 level for fourteen additional chromosomal regions (refer to Table 5).

TABLE 5
Whole genome scan of 5 quantitative immune system phenotypes (CD4+ T
cell, CD8+ T cell, B cell and NK cell numbers, and CD4:CD8 ratio). Summary of (i)
nominal significance levels (P values < 0.01) without covariates by chromosome
regions using multipoint “new Haseman-Elston” weighted regression method; (ii)
corresponding nominal significance levels with both age and sex in the model; and (iii)
empirical P values calculated under the most significant model with or without
covariates. W3 estimates.

| | | | Nominal P | Nominal | |
|---|---|---|---|---|---|
| | | | values | P values | Empirical |
| | | Region | (no | (with | P values |
| Chromosome | Phenotype | (cM) | covariates) P < 0.01 | age + sex) | (best model) |
| 1 | Lymphocyte | 198-218 | 0.0050 | 0.00032 | |
| | count | | | | |
| 1 | CD19 | 197-254 | 0.0013 | 0.00035 | 0.0023 |
| 1 | CD4 | 126-234 | 0.0026 | 0.0052 | |
| 1 | CD8 | 197-218 | 0.00055 | 0.000037 | |
| 2 | Lymphocyte | 248 | 0.0098 | 0.211 | |
| | count | | | | |
| 2 | CD19 | 50-54 | 0.0042 | 0.00053 | |
| 2 | CD4 | 25-37 | 0.0040 | 0.00050 | |
| 2 | CD4:CD8 | 82-94 | 0.0020 | 0.0012 | |
| 3 | NK | 190-200 | 0.0004 | 0.0004 | |
| 4 | CD4 | 31-55 | 0.0034 | 0.020 | 0.0201 |
| 4 | CD8 | 1-3 | 0.0046 | 0.0012 | 0.0050 |
| 4 | CD4:CD8 | 1-3 | 0.00083 | 0.0038 | 0.014 |
| 4 | CD4:CD8 | 53-86 | 0.0000070 | 0.00000019 | <0.000001 |
| 8 | CD4 | 65-68 | 0.0015 | 0.0031 | |
| 9 | CD19 | 102-103 | 0.0077 | 0.00027 | |
| 9 | CD4 | 72-91 | 0.00014 | 0.00024 | |
| 10 | NK | 83-93 | 0.0056 | 0.0054 | |
| 10 | NK | 113-117 | 0.0073 | 0.0071 | |
| 11 | Lymphocyte | 19-42 | 0.0004 | 0.0004 | |
| | count | | | | |
| 11 | CD4 | 22-30 | 0.0051 | 0.020 | |
| 11 | CD8 | 13-138 | 0.0019 | 0.004 | |
| 12 | CD19 | 14-159 | 0.00022 | 0.000083 | |
| 12 | CD4:CD8 | 95-96 | 0.0080 | 0.0053 | |
| 12 | CD4:CD8 | 128-133 | 0.0070 | 0.011 | |
| 18 | Lymphocyte | 90-115 | 0.00032 | 0.031 | |
| | count | | | | |
| 18 | CD19 | 91-96 | 0.0035 | 0.0021 | 0.0049 |
| 18 | CD4 | 90-110 | 0.0000058 | 0.00066 | |
| 18 | CD8 | 114 | 0.0076 | 0.19 | |
| 18 | CD4:CD8 | 115-119 | 0.005 | 0.02 | |

Furthermore, the degree of usefulness of a genetic test in predicting a biological effector disease predisposition is proportional to the amount of the variance in the phenotype (biological parameter) which the individual genetic marker predicts. A test predicting a higher proportion of trait variance will be more useful than one predicting less.

FIG. 1 shows that the QTL which is located between 53-86 cM on the Marshfield map (flanked by D4S405 and D4S2363) predicts up to 47% of the genetic trait variance of CD4 to CD8 T cell ratio. Thus fine mapping and isolating the factor of factors accounting for this effect would have considerable power.

Quantative TDT analysis

As an initial attempt at fine mapping we analyzed data on 34 markers from chromosomes 1, 4, 12 and 18 where significant linkage below the 0.01 level had been established using SIBPAL2. Co-variates were not included in this analysis. Significant results were found for the traits lymphocyte count, CD4 level and CD8 level and are given in Table 6. The use of multiple siblings in this phase means that the test is not a true test of association but establishes association in the presence of linkage for specific alleles with the given phenotypes. Allele 5 on chromosome 12 was significantly associated with lymphocyte count, CD4 level and CD8 level while allele 1 of PLA2 on the same chromosome showed a significant relationship with lymphocyte count and CD4 level (Table 6).

TABLE 6
Results of quantitative transmission
disequilibrium test (QTDT) analysis of
genetic markers/phenotype
combinations on chromosome 1, 4, 12, and 18 where
significant linkage was established
in the genome-wide scan at the p < 0.01 level. A
total of 34 marker/phenotype “events” were tested.
Statistically significant results are
shown after Bonferroni correction
of p values for number of alleles tested.

| | | | | |
|---|---|---|---|---|
| 1Trait | Chromosome | Marker | Allele | p-value |

| | | | | |
|---|---|---|---|---|
| Lymphocyte Ct | 1 | GATA23B04 | 4 | 0.00454 |
| | 12 | GAAA1C01 | 5 | 0.00106 |
| | 12 | PLA2 | 1 | 0.04636 |
| | 12 | GATA10C07 | 1 | 0.04301 |
| Cd4 | 12 | GAAA1C01 | 5 | 0.01132 |
| | 12 | PLA2 | 1 | 0.02338 |
| Cd8 | 12 | GAAA1C01 | 5 | 0.01024 |
| | 18 | AFM193YF8 | 2 | 0.03983 |

All the markers given in Table 6 are given in the CEPH database.

In Table 6, the AFM193YF8 marker shows association in the presence of linkage with CD8 levels. The marker is at 105.02 cM on the Marshfield map (Golden Path position 72537076 and is within 200 kb of candidate gene DNAM-1 (aka CD226—position 72631082-72918927) which has a role in the cytotoxic function of lymphocytes—NB most CD8+ T cells are cytotoxic). The marker is also 500 kb from CIS4, which contains an SH2 domain, and is a gene involved in intracellular signaling and is typical of the genes active in T cells.

Linkage and association analysis are directed towards identifying candidate genes whose variation accounts for the variation in phenotype. The chromosome 18 region linked to lymphocyte count, CD4+ T cell and CD19 B cell levels (cM 90-110) contains a number of candidate genes involved in lymphocyte survival and function which are worthy of investigation. As a first step we genotyped an STR polymorphism associated with the Bc1-2α gene in the CEPH families. Six alleles were observed in the families. Significant transmission distortion was seen for allele 6 with lymphocyte count and CD4+ T cell level indicating association in the presence of linkage. See results given in Table 7.

TABLE 7
Association of Bcl-2 STR alleles with
lymphocyte count and CD4+ T cell
levels. Transmission distortion was
assessed for Bcl-2 alleles against lymphocyte
count, CD4+ T cell and CD19 B cell levels.
Significant transmission distortion was
seen for allele 6 for lymphocyte count and
CD4+ T cell level indicating association in
the presence of linkage.

| | | | | p-value with |
|---|---|---|---|---|
| | | | | Bonferroni |
| Allele | # of Alleles | Sample Size | p-value | Correction |
| Lymphocyte count | | | | |
| 1 | 6 | 9 | 0.79635 | 0.98729 |
| 2 | 6 | 15 | 0.40415 | 0.42855 |
| 3 | 6 | 37 | 0.55637 | 0.99998 |
| 4 | 6 | 0 | | |
| 5 | 6 | 17 | 0.03490 | 1.00000 |
| 6 | 6 | 7 | 0.00000 | 0.00000 |
| CD4+ T cell count | | | | |
| 1 | 6 | 9 | .79201 | .99100 |
| 2 | 6 | 15 | .52136 | .89035 |
| 3 | 6 | 37 | .61164 | .94142 |
| 4 | 6 | 0 | | |
| 5 | 6 | 17 | .18058 | .44981 |
| 6 | 6 | 7 | .00128 | .00383 |

B and T cells form the major components of the adaptive arm of the immune system. In recent years there has been an explosion in the understanding of the molecular basis of many of the components of the immune system. Studies of animal models and individuals who have naturally occurring deficiencies of the immune system along with improvements in molecular biology techniques have led to the identification of many of the genes which are involved in the development and function of the immune system. These include genes responsible for lymphocyte subset differentiation, cell function, immunity to various micro-organisms, and lymphocyte activation regulation. Such studies reveal a complex set of developmental pathways which result in the generation of a large repertoire of B and T lymphocytes, natural killer cells, monocytes, macrophages and dendritic cells. Together, these provide the individual with the capability of mounting successful adaptive and innate immune responses. There are a number of genes characterised whose lack of function result in a total absence of a particular cell type and thus result in a number of immune deficiencies in humans. In contrast, there is little knowledge of how genes may play a role in modulating the actual levels of the individual cell populations whilst still maintaining a functional balance of the immune system.

The extent to which polymorphic genes account for the variation in levels of functional populations of lymphocyte which comprise the human immune system has not been investigated substantially. Several studies have concentrated on T cell populations showing that variation in the important clinical parameters of CD4 and CD8+ T cell levels and CD4:CD8 ratio are significantly heritable. It has also been suggested by segregation analysis that CD4:CD8 ratio and CD4 and CD8+ T cell levels are under major recessive gene control. In the present invention we have used extended families to provide further heritability estimates for variation in the key immune cell populations of CD4, CD8, CD19 B and NK cells and have used a 5 cM genetic map to discover QTLs accounting for significant amounts of the associated genetic variation. A chromosome 4 QTL accounts for up to 40% of the genetic variation associated with human CD4:CDS ratio corroborating the earlier segregation findings. Other QTLs explain lesser proportions of genetic variance for this and other parameters. These data enable the fine mapping and identification of positional candidate genes explaining the observed variation. In order to fine map the genomic regions identified we carried out a transmission disequilibrium test for association in the presence of linkage using allelic markers from 34 loci from chromosomes 1, 4, 12 and 18 where significant linkage below the p=0.01 level had been established in the genome screen. Several associations were observed including a number at adjacent loci suggesting that significant linkage disequilibrium may extend over these regions. Examination of the physical maps within these regions suggests a number of positional candidate genes which can be investigated further. We examined the chromosome 18 region in some detail which includes TCF-4 (56881247-57294117, nucleotide positions in draft sequence according to Golden Path April 2001 freeze (http://genome.ucsc.edu/), IDDM6 (Merriman et al., Diabetes, 50 184-194, 2001), Bc1-2α (65421315-65641279, nucleotide positions as indicated above), Bc1-2α (65613484-65614394, nucleotide positions as indicated above) and RANK, all of which have been implicated in T cell function and/or autoimmune disease risk. In particular, Bc1-2α is recognised as having a profound effect on lymphocyte survival and through associated variation, could be a strong candidate for determining heritable differences in lymphocyte subset levels. To test this idea we identified a polymorphic STR within the first intron of the Bc1-2 gene and genotyped it in the 17 families. Significant transmission distortion was seen at this locus for allele 6 for the correlated measures of lymphocyte count and CD4+ T cell level indicating association in the presence of linkage. Further work will be necessary to exhaustively identify variation at this QTL and to examine its prediction of lymphocyte and CD4+ T cell levels in unrelated individuals. The genetic evidence may also be viewed in light of the fact that Bc1-2α has profound oncogenic potential and that mouse knockout studies show a key role of the gene in T cell development from hematopoietic stem cells.

Do mouse linkage studies help us in interpreting the significance of our findings in humans? Generally they do not, except to demonstrate that analogous variation is under polymorphic genetic control. There is a relative lack of information on lymphocyte subset level variation between inbred experimental mouse strains. The data which do exist are on CD4 and CD8+ T cell levels and CD4:CD8 ratio. Mouse mapping studies for the CD4:CD8 ratio show linkage to the H-2 and TCR alpha regions in some mouse crosses. However, the syntenic regions of chromosomes 6p21 and 14q32 did not contain prominent QTLs in the human screen (data not shown). This suggests profound differences in the way in which variation in this immune parameter and perhaps others, are regulated between humans and highly selected inbred mouse strains.

Studies of the impact of genetic variation on the functioning of the immune system have up to now largely concentrated on qualitative issues of response and non-response. Most studies have been conducted in the mouse, and experimental manipulation of the murine genome using “knock out” and transgenic technologies has enabled insight to be gained in systems which are largely driven to phenotypic extremes. Recently, improvements in the mapping of QTLs for complex traits have been described which enable rapid interval mapping between inbred mouse strains (Grupe 2001). Studies in humans are less advanced. In this study we mapped a number of QTLs for variation in multiple immune system parameters using the core set of CEPH families previously used for construction of the human genetic map. The logic of this approach is that a core set of human subjects organized into extended families can be used efficiently for mapping variation which determines multiple human quantitative traits. The approach is suitable for investigating heritable variables across the normal range and contrasts with the popular strategy of linkage analysis of disease traits in families with multiple affected individuals. This new approach establishes a genetic window on the range of variation associated with “normal” human physiology and function with the aim of understanding the role of selection in shaping the complex network of biological trait variation. Heritabilities of around 50% fall in the middle of the range for human biological variables and it is encouraging that major QTLs have been found to exist for some of these parameters. The precision of cytographic measurements supports accurate heritability estimates by minimising experimental error and in turn increases the power of gene mapping studies.

The extremes of normal variation can represent risk factors for diseases. The QTL which predicts CD4:CD8 ratio on chromosome 4 for example, can have a profound effect on the rate of progression of HIV disease in an untreated subject. This and other QTLs which determine levels of lymphocyte subsets are likely to impact upon human health in a variety of ways.

The method of the present invention opens the way to the investigation of a new class of genetic markers which predict lymphocyte subpopulation levels in cancer, infection and autoimmune disease. The use of an integrated human gene mapping approach for heritable molecular phenotypes in large pedigrees should be considered as a powerful tool for unraveling the effects of polymorphic genes in human health and disease.

In a further experiment the corticotrophin-releasing factor (CRH) was found to be linked to variations in CD4 levels. A QTL analysis, as defined above, was performed based on the presence of a micro satellite marker within 30 kb of the CRH locus (see Fife et al, Arthritis and Rheumatism, 43, 1673, 1678, 2000) with the lymphocyte phenotypes discussed above. The results obtained are shown below in Table 8.

TABLE 8

| Variable | p-value (standard error) |
|---|---|
| CD19 | p = 0.071 (0.0009) |
| CD4 | p = 0.018 (0.0072) |
| CD4/8 | p = 0.266 (0.0677) |
| CD8 | p = 0.768 (0.0031) |
| NK | p = 0.640 (0.0003) |
| Lymph Count | p = 0.1504 (0.0261 |

It can clearly be seen that the presence of the CRH locus effects CD4 levels. The method of QTDT described herein can therefore be used to identify genes which effect biological phenotypes. It should also be noted that the data indicated above indicates that the CRH locus may have an effect on CD19+ cells.

All documents referred to in this application are incorporated herein by reference.

1. A method for identifying genomic regions comprising one or more genes which affect a biological phenotype, comprising performing linkage analysis on one or more extended families wherein the total number of individuals is at least 50.

2. The method according to claim 1, wherein the biological phenotype is the level of lymphocyte subpopulations.

3. The method according to claim 2, wherein the biological phenotype is the level of CD4+ T cells, CD8+ T cells, B cells, NK cells or the ratio of CD4 to CD8+ T cells.

4. The method according to any one of the previous claims wherein the linkage analysis is quantitative trait linkage analysis.

5. The method according to claim 4, wherein the quantitative trait linkage analysis is performed using the SIBPAL2 program implemented in the SAGE package.

6. The method according to any one of the previous claims, wherein the linkage analysis is performed on at least 4 extended families.

7. The method according to any one of the previous claims, wherein the linkage analysis is performed on at least 17 extended families.

8. The method according to any one of the previous claims, wherein the extended families comprise at least 2 generations and at least 5 siblings in the youngest generation.

9. The method according to claim 8, wherein the extended families comprise at least 3 generations.

10. The method to any one of the previous claims, additionally comprising performing fine mapping techniques on the genomic region.

11. The method according to claim 10 where in the fine mapping technique is quantitative transmission disequilibrium test (QTDT) analysis

12. An isolated genomic region identified using the method of any one of claims 1 to 11.

13. An isolated genomic region according to claim 12 which is listed in Table 5.

14. An isolated genomic region from human chromosome 1, which is located at 197 to 218 cM on chromosome 1.

15. The isolated genomic region of claim 14, wherein the genomic region contains one or more genes that affect the level of CD8+ T cells in a human.

16. An isolated genomic region from human chromosome 4, which is flanked by markers D4S405 and D4S2363 on chromosome 4.

17. The isolated genomic region of claim 16 wherein the genomic region contains one or more genes that affect the ratio of CD4 to CD8+ T cells in a human.

18. An isolated genomic region from human chromosome 18, which is located at 90 to 110 cM on chromosome 18.

19. The isolated genomic region of claim 17, wherein the genomic region contains one or more genes that affect the level of CD4+ T cells in a human.

20. A gene contained in the genomic region according to any one of claims 10 to 17, wherein the gene affects the biological phenotype.

21. The gene of claim 20, wherein the gene is locayed within ±1 cM of a marker of the biological phenotype, wherein the marker has a p-value of less than 0.05 as calculated by QTDT analysis.

22. The gene of claim 21, wherein the marker is any one of the markers listed in Table 6.

23. The encoded product of the gene of any one of claims 20 to 22.

24. Use of a probe for the gene of any one of claims 20 to 22, in an assay for detecting an individual's risk of developing a disease, for diagnosing a particular disease, for prognosis of a particular disease or for determining the efficacy or toxicity of a particular treatment.

25. The use of claim 24, wherein the probe is a labelled nucleic acid molecule capable of specifically binding to gene.

26. Use of the gene of any one of claims 20 to 22 in an assay for identifying an agonist or antagonist of the gene.

27. Use of an agonist or antagonist identified by the use of claim 26 in the treatment or prophylaxis of diseases which are caused by the gene.

28. Use of a nucleic acid molecule comprising the gene of any one of claims 20 to 22 in the manufacture of a medicament for use in gene therapy.

29. Use of the encoded product of claim 23 in therapy.

30. Use of the gene of any one of claims 20 to 22 in an assay for identifying a biochemical pathway which is involved in the development or prevention of a disease.

31. A method of predicting the speed of development of AIDS in an individual infected with HIV comprising detecting the presence of one or more genes, which affect the ratio of CD4+ T cells to CD8+ T cells, in the genomic region of chromosome 4 that is flanked by markers D4S405 and D4S2363 in an individual.

32. The method according to claim 27, which comprises:
taking a cell sample from the individual; and
determining the presence of the one or more genes, which increase the ratio of CD4+ T cells to CD8+ T cells,
wherein the presence of the one or more genes is indicative of a genetic predisposition to a high CD4:CDS ratio which reduces the speed of development of AIDS in the individual.
